(19) United States
(12) Patent Application Publication
(10) Pub. No.: US 2026/0035649 A1
(43) Pub. Date: Feb. 5, 2026

(54) BIOREACTOR SYSTEM WITH GASSING INSTALLATION

(71) Applicant: Sartorius Stedim Biotech GmbH, Goettingen (DE)

(72) Inventors: Marco LEUPOLD, Goettingen (DE); Bernward HUSEMANN, Goettingen (DE); Robert STEINICKE, Goettingen (DE); Sören BECKER, Goettingen (DE); Thorsten ADAMS, Goettingen (DE)

(21) Appl. No.: 19/104,415

(22) PCT Filed: Jul. 20, 2023
(86) PCT No.: PCT/EP2023/070228

§ 371 Date: Feb. 18, 2025

(30) Foreign Application Priority Data
Aug. 19, 2022 (EP) 22191238.9

(51) Int. Cl. C12M 1/34; C12M 1/00; C12M 1/06; C12M 1/36

(52) U.S. Cl. CPC C12M 41/34; C12M 23/40; C12M 27/02; C12M 29/06; C12M 41/48

(57) ABSTRACT

A bioreactor system for carrying out a biological process including a vessel for receiving a liquid biological medium, and a gassing installation for controlled supply of different gases from gas sources into the vessel. The gassing installation has a plurality of gas outlet lines which open into gassing devices and/or into an overlay gas outlet in the interior of the vessel. Each gas outlet line is connected to a plurality of gas sources via a respective gas supply line or via a respective branch from a gas supply line. A mass flow controller connected to a controller is arranged in each gas supply line and in each branch. The system may include a controller with a master controller for a controlled variable, at least one sensor associated with the master controller, and one or more slave controllers having actuators, the manipulated variables of which purposefully influence the controlled variable.

### FIELD OF THE INVENTION

The invention relates to a bioreactor system for carrying out a biological process having a gassing installation.

### BACKGROUND

In the present case, a bioreactor system is generally understood to mean an apparatus having a vessel by means of which biotechnological processes, such as biopharmaceutical processes can be carried out or supported. In addition to bioreactors in which microorganisms or mammalian cells are cultured under certain conditions, process mixing equipment by means of which liquid biological media such as suspensions, solutions, emulsions or similar can be mixed is a further example. The vessel of the bioreactor system may be a dimensionally stable vessel or a flexible bag.

A bioreactor system may be equipped with one or more gassing devices (spargers) to introduce air and/or other gases directly into a liquid biological medium present in the vessel. In the biotechnological context of cell cultivation, gassing controls the introduction of air or gas into a nutrient broth inside the vessel to create an optimal environment for cell growth. Therefore, in contrast to some bacterial cultures, cell cultures are not only gassed with air, but with oxygen (O₂), nitrogen (N₂), carbon dioxide (CO₂) and air in an optimized composition for the respective cell type and fermentation. Since the pH value in the medium changes during cell growth, but a constant pH value is essential for many production processes, this must also be regulated. Regulation is usually carried out via controlled gassing with CO₂.

Typically, gassing rings or discs are used as gassing devices, or a gas is introduced directly into the liquid biological medium via a gas outlet line. There are also combined gassing devices which include a housing having two gassing channels inside. One or more gases can be respectively introduced into an assigned gassing channel of the housing via two gas inlet openings in the housing. The first gassing channel is assigned a plurality of larger gas outlet openings having a diameter of more than 0.2 mm, e.g. 0.8 mm, while the second gassing channel is assigned a plurality of smaller gas outlet openings having a smaller diameter, e.g. 0.15 mm or smaller. The combined gassing device can thus selectively be used as a so-called “ring sparger” utilizing the larger openings or as a “microsparger” utilizing the smaller openings.

In addition, a gas can be supplied to the bioreactor vessel from the top, or more specifically into the space above the medium. Such a so-called “overlay” or head gassing can be carried out for various reasons, among others to ensure that the bag remains inflated when using a bioreactor system having a flexible bag.

To supply different gases into the bioreactor vessel, a gas supply station is usually provided with gas lines leading from the corresponding gas sources to the gas outlets in the vessel, i.e. to the ring sparger and microsparger, if provided, and to an overlay gas outlet, the ring sparger and the microsparger sharing one gas line. Mass flow controllers can be used to control the gas quantity supplied, and shut-off valves can be used to switch the flow paths (from the respective gas source to the desired gas outlet). In such a conventional configuration, it must be decided in advance for a particular gas whether it is supplied via the ring sparger, via the microsparger or to the overlay gas outlet. The respective flow path is released by switching the shut-off valves accordingly. However, this limits the possibilities for gassing.

### SUMMARY

The object of the invention is to enable a more flexible supply of gas into a bioreactor vessel.

This object is achieved by a bioreactor system having the features of claim 1. Advantageous and practical configurations of the bioreactor system according to the invention are specified in the sub-claims.

The bioreactor system according to the invention for carrying out a biological process comprises a vessel for receiving a liquid biological medium, and a gassing installation for controlled supply of different gases from gas sources into the vessel. The gassing installation has a plurality of gas outlet lines which open into one or more gassing devices and/or into an overlay gas outlet in the interior of the vessel. Each gas outlet line is connected to a plurality of gas sources via a respective gas supply line or via a respective branch from a gas supply line. A mass flow controller is connected to a control unit is arranged in each gas supply line and in each branch.

The configuration of the gassing installation of the bioreactor system according to the invention thus provides that each connection between a gas source and a gas outlet line, via which gas is supplied into the bioreactor vessel, is provided with its own mass flow controller. The gas outlet lines of the gassing installation are typically connected to gassing devices in the form of spargers, in particular a ring sparger and a microsparger or a combined sparger, and/or to an overlay gas outlet. Therefore, each sparger and each overlay gas outlet can be supplied individually with gas. The provision of individual mass flow controllers in all connections also makes it possible to conduct one gas type (e.g. oxygen) simultaneously and individually in a regulated manner via several spargers, in particular a ring sparger and a microsparger and optionally further spargers (of other types), and/or further gas outlet lines into the bioreactor vessel. It is also possible to mix different gases in variable proportions.

Further advantages of such a bioreactor configuration are a more precise and finer regulation of the gas supply, which allows a purposeful adjustment of the bubble size and the number of bubbles, and a gas saving.

In a preferred embodiment of the bioreactor system according to the invention, a first gas outlet line which opens into a ring sparger is connected to an air source and/or an oxygen source and/or a nitrogen source and/or a carbon dioxide source. A second gas outlet line which opens into a microsparger is preferably connected to an air source and/or an oxygen source. A third gas outlet line which opens into an overlay gas outlet is preferably connected to an air source and/or a carbon dioxide source.

According to an independent aspect of the invention, a bioreactor system for carrying out a biological process, in particular a bioreactor system of the type described above, includes a controller which comprises a master controller (primary regulator) for a controlled variable (regulating variable), at least one sensor associated with the master controller, and one or more slave controllers (follow-up regulators) having actuators, the manipulated variables of which purposefully influence the controlled variable. The controller is set up such that a setpoint value for the controlled variable can be specified for the master controller, the at least one sensor is used to repeatedly determine an actual value of the controlled variable, and the master controller outputs an output signal to the slave controllers, which depends on the deviation of the actual controlled variable value from the setpoint controlled variable value. Control profiles which depend on the output signal of the master controller are assigned to the slave controllers. According to a first alternative, the control profiles contain setpoint values for the actuators of the slave controllers, and the controller is set up such that the setpoint values for the actuators can be entered in the physical unit of the manipulated variables thereof. According to a second alternative, the control profiles contain setpoint values for a parameter which is directly related to the biological process and can be purposefully influenced by the actuators, and the controller is set up to automatically assign setpoint values for the actuators of the slave controllers to the parameter setpoint values.

Due to the controller set up in accordance with the invention, the user does not have to think in generic units such as percentage values of output signals from slave controllers and enter correspondingly abstract data.

Rather, according to the first alternative of the invention, the user can enter real physical values for the setpoint values of the actuators, such as a stirrer arranged in the bioreactor vessel and mass flow controllers of a gassing installation, without having to consider which output value of the slave controller corresponds to a certain stirrer speed or a gassing rate.

According to the second alternative of the invention, the entry of the user is even less abstract, namely such that the user specifies a setpoint value for a process-related parameter, i.e. a parameter characterizing the process, e.g. a desired value for the volume-related mass transfer coefficient (k_{L}a value). The controller then automatically applies the control profiles assigned to this value to control the actuators of the slave controllers, in particular the stirrer and the mass flow controllers. This is particularly advantageous if the user has developed a bioprocess on a small laboratory scale and does not know how to adjust the actuators of a large bioreactor system to achieve and maintain the same parameter value when scaling up to a large production scale. This is carried out automatically by the controller which is adapted to the large bioreactor system; the user only has to enter the desired parameter value.

Apart from these advantages, the invention contributes significantly to reducing the risk of incorrect entries.

A process-related parameter value which the user enters instead of percentage values for the slave controllers is in particular a value for one of the following parameters: cell growth rate, cell density, oxygen transfer rate, oxygen intake rate, bubble size, bubble number, mixing time, volume-related mass transfer coefficient.

From the user's point of view, these parameters are important parameters which characterize his process. For example, the bubble size has a significant influence on how the oxygen is distributed in the bioreactor vessel. With the small bubbles generated by a microsparger, the surface tension is the dominant force. They therefore have a long residence time in the reactor, which improves oxygen mass transfer (oxygen input), but makes them less suitable for stripping carbon dioxide from the culture. The larger bubbles generated by a ring sparger have a shorter residence time but dissolve less easily than smaller bubbles. They serve in particular for CO₂ stripping.

The dissolved oxygen is particularly suitable as a controlled variable for the master controller of the bioreactor system, as a corresponding sensor is usually always present. However, the controlled variable may basically also be another parameter which can be measured directly using the at least one sensor, such as one of the following: carbon dioxide content, cell number, cell density, living cell volume, turbidity, glucose content, pH value, carbon dioxide partial pressure, oxygen partial pressure.

According to a further development of the invention, the controlled variable may even be a parameter which cannot be measured directly using the at least one sensor but can be determined by means of the at least one sensor, such as one of the following: oxygen transfer rate, oxygen intake rate, cell growth rate, glucose consumption rate, carbon dioxide production rate, carbon dioxide discharge rate, respiratory quotient, biomass yield, glucose yield, yield of another carbon or nitrogen source. To determine the actual value of the parameter, additional calculations are carried out and/or a previously created mathematical model is applied. In such a case, the sensor and the necessary aids may be collectively referred to as a “soft sensor”.

In a preferred embodiment of the bioreactor system according to the invention, the actuators comprise a stirrer arranged in a vessel of the bioreactor system and a plurality of mass flow controllers of a gassing installation. In accordance with the first alternative of the invention, the controller may then be set up such that values in the unit rpm (revolutions per minute) can be entered as setpoint values for the stirrer and values in the unit L/min (liters per minute) can be entered as setpoint values for the mass flow controllers. Of course, this should include units derived or derivable therefrom such as rps (revolutions per second) or mL/sec (milliliters per second), etc., as these are still physical units of the manipulated variable of the stirrer or the mass flow controllers—in contrast to percentage values based on the control range (regulating range) of the respective slave controller.

The control profiles assigned to the slave controllers and depending on the output signal of the master controller may be stored directly in the controller or in an external database which the controller can access. The control profiles themselves may be specified by the manufacturer or the user, or they can be individually preset by the user.

Preferably, the control profiles are based on empirically determined values or on a previously created mathematical model.

A further development of the invention provides that the controller monitors the entries of the setpoint values for the actuators of the slave controllers, in particular the stirrer and the mass flow controllers, to the effect that no setpoint value combinations are entered which would lead to improper operation of the bioreactor system. It is thus possible to avoid in particular flooding behavior or unnecessarily high power inputs without gassing.

In addition, the controller can be used to automatically specify the time sequence (start time) and the driving of the respective actuators. For example, higher k_{L}a values and a more even bubble distribution can be achieved by first starting the gassing and then the stirrer.

### BRIEF DESCRIPTION OF THE DRAWINGS

Further features and advantages of the invention will become apparent from the following description and the accompanying drawings, to which reference is made and in which:

FIG. 1 shows a bioreactor system with a gassing installation;

FIG. 2 shows a greatly simplified representation of a gassing device;

FIG. 3 shows a circuit diagram of a conventional gas supply control of a bioreactor system;

FIG. 4 shows a circuit diagram of a gas supply control of a bioreactor system according to the invention;

FIG. 5 shows a plurality of control profiles adapted to a controlled variable for driving various actuators during a bioprocess.

### DETAILED DESCRIPTION

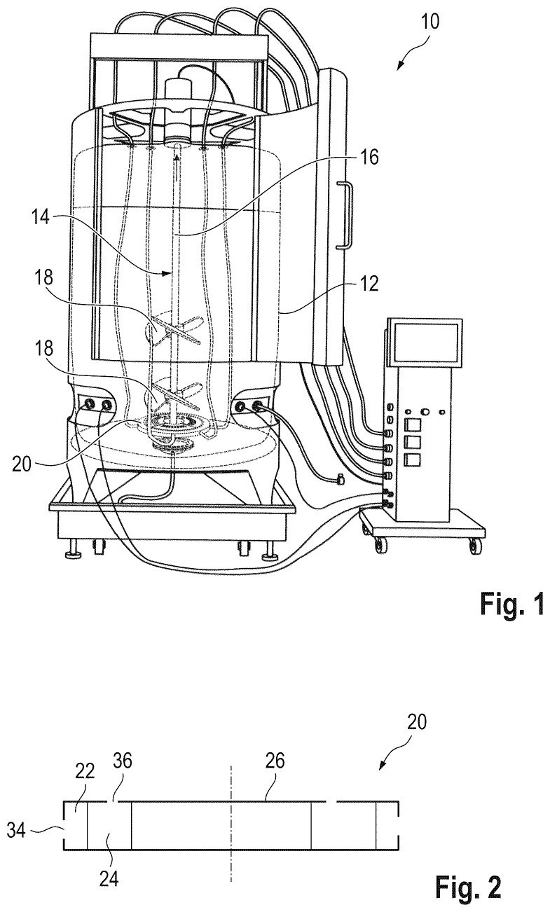

FIG. 1 shows an exemplary embodiment of a bioreactor system 10 for culturing cells, which comprises a vessel 12, here in the form of a flexible disposable bag. The vessel 12 is intended to receive a substantially liquid biological medium serving as a nutrient medium.

The bioreactor system 10 further comprises a stirrer 14 installed in the interior of the vessel 12. The stirrer 14 has a stirrer shaft 16 on which one or more stirrer elements 18 are arranged. The stirrer 14 is used in particular to mix the biological medium during culturing.

As also shown in FIG. 1, the bioreactor system 10 further comprises a gassing device 20 arranged in the vessel 12, which serves to supply several gases to the biological medium, in particular oxygen, carbon dioxide, nitrogen and air.

As can be seen in schematic FIG. 2, the gassing device 20 has a plurality, here two, separate gassing channels 22, 24, which are formed within a housing 26. A gas inlet is formed in the housing 26 for each gassing channel 22, 24, via which a gas can be introduced into the respective gassing channel 22, 24. In the assembled and operational state, each gas inlet is in fluid communication with a corresponding gas source via a corresponding gas outlet line 28, 30.

Furthermore, the housing 26 has a plurality of gas outlet openings 34, 36, via which the gas can be discharged from the respective gassing channel 22, 24 into the liquid biological medium surrounding the gassing device 20 in the interior of the vessel 12.

In the illustrated example embodiment, the outer first gassing channel 22 has larger gas outlet openings 34 having a diameter of more than 0.2 mm, preferably from 0.25 to 4.0 mm, or a cross-sectional area of more than 0.03 mm², preferably from 0.05 to 12.0 mm², and corresponds to an ring sparger. The inner second gassing channel 24 has smaller gas outlet openings 36 having a diameter of at most 0.2 mm, preferably from 0.005 to 0.2 mm, or a cross-sectional area of at most 0.03 mm², preferably from 0.00002 to 0.03 mm², and corresponds to a microsparger. For the sake of simplicity, the first gassing channel 22 is referred to as ring sparger 22 and the second gassing channel 24 as microsparger 24 in the following.

In addition to the gassing device 20, an overlay gassing (not shown in FIG. 1) is provided, in which air or carbon dioxide can be supplied via a further gas outlet line 32 opening into the space above the liquid biological medium into a gas outlet.

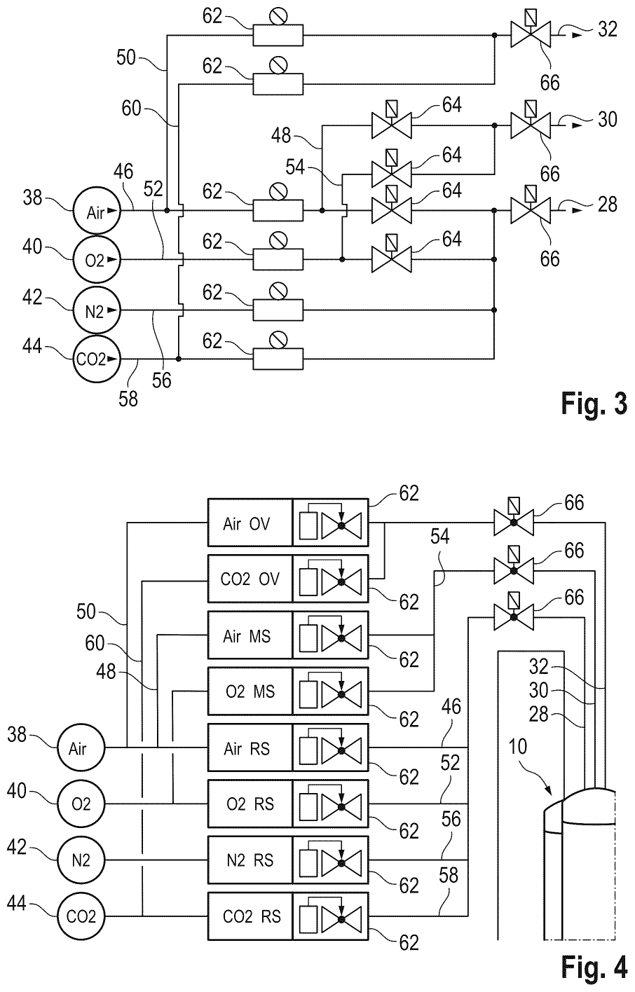

FIG. 3 shows an example of a circuit diagram of a conventional gas supply station having four different gas sources 38, 40, 42, 44 (air, oxygen, nitrogen and carbon dioxide).

An air supply line 46 leads from the air source 38 to the first gas outlet line 28, which opens into the ring sparger 22. A first branch 48 leads from the air supply line 46 to the second gas outlet line 30, which opens into the microsparger 24. A second branch 50 leads from the air supply line 46 to the third gas outlet line 32, which opens into the overlay gas outlet.

An oxygen supply line 52 leads from the oxygen source 40 to the first gas outlet line 28, which opens into the ring sparger 22. A third branch 54 leads from the oxygen supply line 52 to the second gas outlet line 30, which opens into the microsparger 24.

A nitrogen supply line 56 leads from the nitrogen source 42 to the first gas outlet line 28, which opens into the ring sparger 22.

A carbon dioxide supply line 58 leads from the carbon dioxide source 44 to the first gas outlet line 28, which opens into the ring sparger 22. A fourth branch 60 leads from the carbon dioxide supply line 58 to the third gas outlet line 32, which opens into the overlay gas outlet.

A total of six mass flow controllers 62 are provided, which are connected to a controller and are arranged at the following points: 1) in the air supply line 46 between the first branch 48 and the second branch 50; 2) in the oxygen supply line 52 upstream of the third branch 54; 3) in the nitrogen supply line 56; 4) in the carbon dioxide supply line 58 downstream of the fourth branch; 5) in the second branch 50; 6) in the fourth branch 60.

Furthermore, according to the circuit diagram of FIG. 3, four switching valves 64 are arranged at the following points: 1) in the air supply line 46 downstream of the mass flow controller 62 and the first branch 48; 2) in the oxygen supply line 52 downstream of the mass flow controller 62 and the third branch 54; 3) in the first branch 48; 4) in the third branch 54. The switching valves 64 are switched alternately as required so that the respective gas is directed either to the ring sparger 22 or to the microsparger 24.

Furthermore, each of the three gas outlet lines 28, 30, 32 can be shut off and released by a shut-off valve 66, typically a solenoid valve.

For example, to achieve a supply of oxygen into the liquid biological medium received in the vessel 12 of the bioreactor system 10 via the microsparger 24, a suitable flow rate must be set at the mass flow controller 62 in the oxygen supply line 52, and it must be ensured that the switching valve 64 arranged downstream of the third branch 54 is closed, while the switching valve 64 in the third branch 54 and the shut-off valve 66 in the second gas outlet line 30 are open.

FIG. 4 shows an example of a circuit diagram for the gas supply of a bioreactor system 10 according to the invention. The layout of the lines 28, 30, 32, 46, 52, 56, 58 and the branches 48, 50, 54 and 60 is basically the same as in the gas supply station described above, which can be designed as a module of the bioreactor system 10. Here, too, shut-off valves 66 are provided to shut off and release the three gas outlet lines 28, 30, 32, but there are no switching valves 64. Instead, a total of eight mass flow controllers 62 connected to a controller are used here, which are arranged in all four gas supply lines 46, 52, 56, 58 from the gas sources 38, 40, 42, 44 and in all four branches 48, 50, 54, 60. This means that one mass flow controller 62 is provided in each gas path.

This configuration makes it possible to individually control the supply of all gases to the ring sparger 22 and the supply of air and oxygen to the microsparger 24 and to the overlay gas outlet. In particular, due to the mass flow controllers 62, it is possible to individually set for each gas whether and at what mass flow it is supplied to the respective gas outlet line 28, 30, 32. It is in particular possible to supply a gas to several gas outlet lines 28, 30, 32 at the same time. For example, oxygen can be supplied simultaneously to the ring sparger 22 and the microsparger 24 at different mass flows.

Of course, it is also possible to provide more or fewer and/or other gas sources. It is also possible to provide more or fewer gas outlet lines which lead into spargers or directly into the liquid biological medium or are intended for overlay gassing. It is important that a separate mass flow controller 62 is provided for each intended gas supply to a gas outlet line.

In a configuration in which, as described by way of example on the basis of FIG. 4, a separate mass flow controller 62 is provided for each gas supply line 46, 52, 56, 58 from a gas source 38, 40, 42, 44 to a gas outlet line 28, 30, 32, and for each branch 48, 50, 54, 60, which also establishes a connection between a gas source 38, 40, 42, 44 and a gas outlet line 28, 30, 32, or in a conventional configuration, as described above by way of example on the basis of FIG. 3, a special controller can be used, which is described below.

A measurement and control system (instrumentation and regulation system) (not shown separately in the figures), hereinafter referred to as “controller” for the sake of simplicity, comprises in the present case a master controller having at least one associated sensor and a plurality of slave controllers having actuators which can basically be driven simultaneously (in this case in particular the stirrer 14 and the mass flow controllers 62). Further sensors can be assigned to the slave controllers.

The at least one sensor which is assigned to the master controller is typically a sensor for measuring the dissolved oxygen (DO sensor). Other possible sensors will be discussed in detail later.

The controller is set up such that, to carry out a bioprocess (or bioprocess step), the user can enter a setpoint value to be kept constant for a controlled variable monitored during the process. In the example described here, the user can thus enter a setpoint value for the dissolved oxygen. The controller has a DO sensor for continuous monitoring (repeated measurements) of this parameter and a controlling means (regulating means) for this controlled variable (DO controlling means), which generates an output signal which depends on the deviation of the actual controlled variable value determined by means of the sensor from the setpoint controlled variable value set by the user.

The DO controlling means works as a master controller, i.e. depending on the configuration, it acts on one or more slave controllers to control actuators which can purposefully influence the DO value in the process. If there is a deviation from the setpoint value, the master controller sends an output signal (reference variable) to the slave controllers, which is relative to the control range (0% . . . 100%). The slave controllers control their actuators (here in particular the stirrer 14 for mixing and the mass flow controllers 62 for gassing) in accordance with this output signal. More specifically, a control profile is stored for each slave controller, which specifies its control behavior (regulating behavior) as a function of the output signal of the master controller.

In the example considered here, several setpoint values (e.g. five) can be assigned to each slave controller, depending on the output signal of the master controller. For example, a setpoint value can respectively be entered for the actuators of the slave controllers for the output values 0%, 10%, 20%, 30%, 40% and 50% of the master controller. Usually, the user will specify these setpoint values. However, the setpoint values can also be specified by the manufacturer and stored in a controller memory. The setpoint values can also be stored in an external database which the controller can access. The setpoint values for the slave controllers are matched to the specific bioreactor configuration and are based on empirical values and/or a mathematical analysis model which has been created by a comprehensive process engineering characterization.

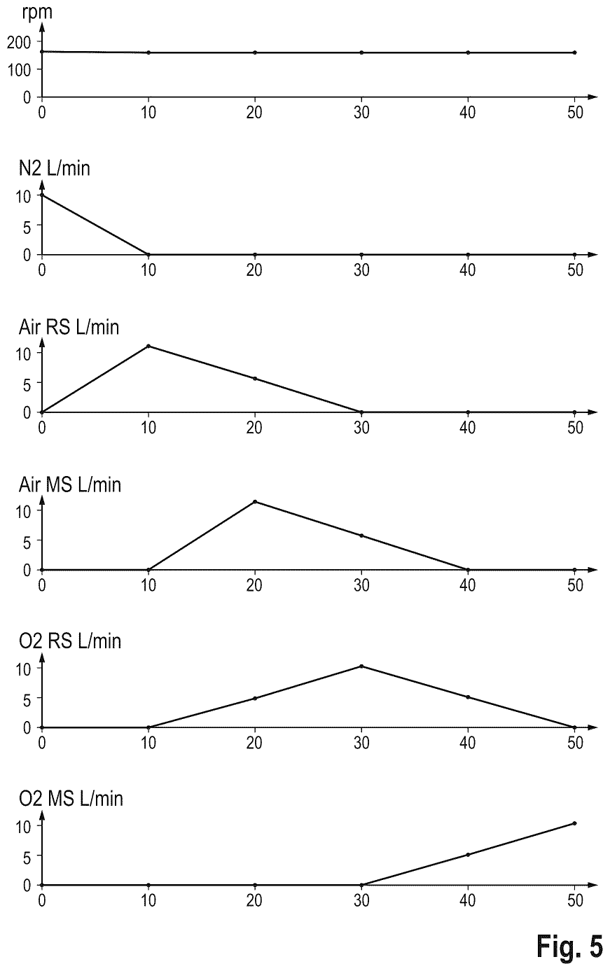

A characteristic feature of the controller is that the setpoint values for the slave controllers do not have to be entered in abstract output values relative to their control ranges (0% . . . 100%). Rather, the setpoint values can be entered in the physical unit of the respective manipulated variable. In the example considered here, the user can thus enter a specific speed (rpm) for the stirrer and specific gassing rates (L/min) for the mass flow controllers 62 for the various output values of the DO controlling means. The setpoint values defined in this way can be plotted graphically as a polygonal chain against the output signal of the master controller, as shown by way of example in FIG. 5, where the x-axis indicates the output of the master controller and the y-axes indicate the associated manipulated variables of the actuators in the physical unit of the respective manipulated variable.

As a safety function of the controller, it may be provided that if setpoint values are entered for the stirrer 14 and the mass flow controllers 62 which would lead to excessive gas input or an excessive stirrer speed and the resulting so-called “impeller flooding” (flooding behavior), a warning is issued, or that the entry of such values is not accepted at all. The same type of reaction can occur for entries which would result in unnecessarily high power inputs without gassing.

With reference to the example considered here, the DO value measured by the DO sensor is repeatedly compared with the setpoint value specified by the user during operation, i.e. during the ongoing process, and the DO controlling means outputs an output signal relative to its control range according to the deviation detected. The slave controllers automatically adjust the stirrer 14 and the mass flow controllers 62 to the setpoint values previously set for this output value of the DO controller, so that the DO setpoint value is maintained.

In the graphical representation of several polygonal chains shown by way of example in FIG. 5, the regulation to a setpoint value specified by the user for the controlled variable is achieved by appropriately driving the stirrer 14 and the mass flow controllers 62 such that the stirrer speed (top diagram) is kept at 160 rpm over the entire control range of interest of the master controller (here 0% to 50%). The addition of nitrogen via the ring sparger 22 (second diagram from the top) is 10 L/min at an initial value of 0% of the master controller and 0 from an initial value of 10% of the master controller, i.e. no nitrogen is added anymore even in the event of larger deviations. Air is supplied both via the ring sparger 22 (third diagram from the top) and via the microsparger 24 (fourth diagram from the top). The respective setpoint values are similar, but offset with respect to the initial values of the master controller. Different polygonal chains are provided for the supply of carbon dioxide via the ring sparger 22 (fifth diagram from the top) and the microsparger 24 (sixth diagram from the top).

According to a further embodiment of the controller, the output values of the master controller are not linked to any setpoint values for the manipulated variables of the actuators, but at least one setpoint value for a parameter which is directly related to the process and which can be purposefully influenced by the actuators. In other words: the user does not assign setpoint values for the stirrer 14 and the mass flow controllers 62 to the various output values of the master controller, but simply enters a respective setpoint value for a parameter which can be controlled by these actuators, such as a desired cell growth rate, cell density, oxygen transfer rate (OTR), oxygen intake rate (OUR), bubble size, bubble number, mixing time or a desired volume-related mass transfer coefficient (k_{L}a value).

The user is usually familiar with such parameters. The k_{L}a value is of great importance in a biotechnological process, for example, as it provides information on how well microorganisms in the vessel 12 of the bioreactor system 10 can be supplied with gases. When culturing aerobic microorganisms, it is important to achieve a high k_{L}a value for oxygen, while for microorganisms growing autotrophically, the k_{L}a values for hydrogen and carbon dioxide also play an important role.

If the user has developed a process on a laboratory scale with a small bioreactor, he knows the optimum values for one or more of these parameters. However, the settings of the actuators required to achieve the desired parameter value, in particular of the stirrer and the mass flow controllers, cannot be transferred 1:1 to a large-volume process in a large bioreactor with different actuators, in particular a larger stirrer 14 and spargers 22, 24. This means that to achieve a desired parameter value, other setpoint values must be specified for the actuators, which the user must first find out.

This task is performed by the controller. The user only enters the desired parameter value as a setpoint value (e.g. a k_{L}a setpoint value) which he knows from the process developed on a laboratory scale, and the controller assigns a control profile to this parameter setpoint value, by means of which the parameter setpoint value is achieved in the large bioreactor on a production scale. The control profile includes setpoint values for the slave controller actuators, as described above. The assignment of these setpoint values for the slave controllers to a desired parameter value is in turn based on an analytical model which has been created through a comprehensive process engineering characterization. For example, setpoint values for the stirrer 14 and the mass flow controllers 62 are respectively stored for various k_{L}a values, which are matched to the present bioreactor configuration. The basis for this is that the k_{L}a value depends, among other things, on the stirrer speed and the contact area with the air (bubble size).

According to a further development of the controller, it is also possible to use a parameter other than the DO value measured by a DO sensor as a controlled variable for controlling the process. In particular, one of the following parameters, which can be directly monitored during the process using the appropriate sensors, can be used as a controlled variable: CO₂ content, cell number, cell density, living cell volume, turbidity, glucose content, pH value, carbon dioxide partial pressure (pCO₂), oxygen partial pressure (pO₂).

Furthermore, it is also possible to use parameters as a controlled variable for controlling the process which cannot be measured directly using a sensor, but can only be calculated from measured values of one or more sensors or determined using a previously created model. These parameters include, among others, the oxygen transfer rate (OTR), the oxygen intake rate (OUR), the cell growth rate, the glucose consumption rate, the carbon dioxide production rate (CER) and carbon dioxide discharge rate, the respiratory quotient (RQ value), the biomass yield, the glucose yield and generally the yield of other carbon or nitrogen sources. The hardware (sensor(s)) and software (algorithms, assignment tables, etc.) required to determine such parameters during the process can be collectively referred to as “soft sensors”.

Alternatively or additionally, it is also possible to take the measured values of an off-gas sensor into account, which, for example, measures the oxygen or carbon dioxide content of the off-gas discharged from the vessel 12 of the bioreactor system 10.

Spectroscopic analyzers (e.g. IR, UV/VIS, Raman spectrometers, etc.) can also be used as “sensors” to determine a controlled variable parameter. Devices for carrying out measurements related to substances which the cells in the bioreactor vessel metabolize or serve as inducers for gene expression (e.g. lactate, methanol/ethanol, etc.) can also be used as sensors.

The controller can furthermore be set up such that the user can set either a heterogeneous or a homogeneous bubble pattern, depending on the application of the parameters. A heterogeneous bubble pattern ensures more effective mixing, while a homogeneous bubble pattern ensures improved gas transfer. The gassing rate (mass flow) and the stirrer speed are automatically adjusted by the controller to achieve the desired bubble pattern.

The control of the process to a controlled variable other than the DO value is basically carried out in the same way as described above, i.e. for a setpoint value for such a controlled variable, setpoint values for the actuators of slave controllers can be entered in the physical unit of the respective manipulated variable or setpoint values can be entered for a parameter which is directly related to the process. In the latter case, the controller automatically assigns suitable setpoint values for the actuators.

Depending on the controlled variable, slave controllers may be provided which have other actuators in addition or as an alternative to the stirrer 14 and the mass flow controllers 62, such as a pump for supplying a medium (e.g. a nutrient solution or a buffer).

The entry of the setpoint values for the controlled variable(s) and the setpoint values for the actuators of the slave controllers, which depend on the output value of the master controller, or a process-related parameter, can be carried out via a software layer which is superimposed on a conventional controller. However, the controller can also be specially designed from the start to process such input setpoint values.

The configurations of the gassing installation and the controller described as examples are basically suitable for all bioreactor sizes.

### List of Reference Numerals

10 bioreactor system
12 vessel
14 stirrer
16 stirrer shaft
18 stirring element
20 gassing device
22 (outer) first gassing channel; ring sparger
24 (inner) second gassing channel; microsparger
26 housing
28 first gas outlet line (ring sparger)
30 second gas outlet line (microsparger)
32 third gas outlet line (overlay)
34 gas outlet openings of the first gassing channel
36 gas outlet openings of the second gassing channel
38 air source
40 oxygen source
42 nitrogen source
44 carbon dioxide source
46 air supply line
48 first branch
50 second branch
52 oxygen supply line
54 third branch
56 nitrogen supply line
58 carbon dioxide supply line
60 fourth branch
62 mass flow controller
64 switching valve
66 solenoid valve

1. A bioreactor system for carrying out a biological process, comprising:
a vessel for receiving a liquid biological medium, and
a gassing installation for controlled supply of different gases from gas sources into the vessel,
the gassing installation having a plurality of gas outlet lines which open into one or more gassing devices or into an overlay gas outlet in an interior of the vessel,
each gas outlet line being connected to a plurality of gas sources via a respective gas supply line or via a respective branch from a gas supply line, and
a mass flow controller connected to a controller being arranged in each gas supply line and in each branch.

2. The bioreactor system according to claim 1, wherein a first gas outlet line which opens into a ring sparger is connected to an air source, an oxygen source, a nitrogen source, or a carbon dioxide source.

3. The bioreactor system according to claim 1, wherein a second gas outlet line which opens into a microsparger is connected to an air source or an oxygen source.

4. The bioreactor system according to claim 1, wherein a third gas outlet line, which opens into the overlay gas outlet, is connected to an air source or a carbon dioxide source.

5. The bioreactor system for carrying out the biological process, according to claim 1, further comprising a controller which comprises a master controller for a controlled variable, at least one sensor associated with the master controller, and one or more slave controllers having actuators, manipulated variables of which purposefully influence the controlled variable,
the controller being set up such that
a setpoint value for the controlled variable can be specified for the master controller,
the at least one sensor is used to repeatedly determine an actual value of the controlled variable, and
the master controller outputs an output signal to the slave controllers, which depends on a deviation of the actual controlled variable value from the setpoint controlled variable value,
control profiles which depend on the output signal of the master controller being assigned to the slave controllers, and
the control profiles containing setpoint values for the actuators of the slave controllers, and the controller being set up such that the setpoint values for the actuators can be entered in a physical unit of the manipulated variables thereof, or
the control profiles containing setpoint values for a parameter which is directly related to the biological process and can be purposefully influenced by the actuators and the controller being set up to automatically assign setpoint values for the actuators of the slave controllers to the parameter setpoint values.

6. The bioreactor system according to claim 5, wherein the parameter is a cell growth rate, a cell density, an oxygen transfer rate, an oxygen intake rate, a bubble size, a bubble number, a mixing time, or a volume-related mass transfer coefficient.

7. The bioreactor system-according to claim 5, wherein the controlled variable is a parameter which can be measured directly by means of the at least one sensor.

8. The bioreactor system according to claim 5, wherein the controlled variable is a parameter which cannot be measured directly using the at least one sensor but can be determined by means of the at least one sensor.

9. The bioreactor system according to claim 5, wherein the actuators comprise a stirrer arranged in a vessel of the bioreactor system and a plurality of mass flow controllers of a gassing installation, and wherein the controller is set up such that values in a unit rpm can be entered as setpoint values for the stirrer and values in a unit L/min can be entered as setpoint values for the mass flow controllers.

10. The bioreactor system according to claim 5, wherein the control profiles are stored in the controller.

11. The bioreactor system according to claim 5, wherein the control profiles are stored in an external database which can be accessed by the controller.

12. The bioreactor system according to claim 5, wherein the control profiles are based on empirical values or on a mathematical model.

13. The bioreactor system according to claim 9, wherein the control unit monitors entries of the setpoint values for the slave controllers, for the stirrer and the mass flow controllers, to ensure that no setpoint value combinations are entered which would lead to improper operation of the bioreactor system.

14. The bioreactor system according to claim 5, wherein the control unit monitors entries of the setpoint values for the slave controllers to ensure that no setpoint value combinations are entered which would lead to improper operation of the bioreactor system.

15. The bioreactor system according to claim 7, wherein the controlled variable is a dissolved oxygen, a carbon dioxide content, a cell number, a cell density, a living cell volume, a turbidity, a glucose content, a pH value, a carbon dioxide partial pressure, or an oxygen partial pressure.

16. The bioreactor system according to claim 8, wherein the controlled variable is an oxygen transfer rate, an oxygen intake rate, a cell growth rate, a glucose consumption rate, a carbon dioxide production rate, a carbon dioxide discharge rate, a respiratory quotient, a biomass yield, a glucose yield, or a yield of a carbon or nitrogen source.
